(19) United States
(12) Patent Application Publication
(10) Pub. No.: US 2003/0236493 A1
(43) Pub. Date: Dec. 25, 2003

(54) Articulating handle for a deflectable catheter and method therefor

(75) Inventor: Grant Mauch, Hopkins, MN (US)

Correspondence Address: SCHWEGMAN, LUNDBERG, WOESSNER & KLUTH, P.A., P.O. BOX 2938, MINNEAPOLIS, MN, 55402, US

(73) Assignee: MedAmicus, Inc.

(21) Appl. No.: 10/179,633

(22) Filed: Jun. 25, 2002

(51) Int. Cl. A61M 31/00

(52) U.S. Cl. 604/95.04

(57) ABSTRACT

A catheter assembly includes a handle assembly, and a catheter body coupled with the handle assembly, where the catheter body extends to a deflectable distal end, and the deflectable distal end is controllable by a flexible element. An actuator member is coupled with the flexible element, and movement of the actuator member provides for movement of the flexible element. The actuator has a locked mode and an operational mode, where the actuator and the flexible element are not movable relative to the handle assembly when the actuator is in the locked mode.

### TECHNICAL FIELD

The present invention relates generally to deflectable catheter assemblies. More particularly, it pertains to an articulating handle for a deflectable catheter.

### BACKGROUND

Increase in the use of stents, leads, and ablation techniques in branch vessels has provided an increased demand in the placement techniques for the devices. For some procedures, it is necessary to initially position a guidewire into a desired part of the lumen of a desired vessel or duct, such as a blood vessel. After the guidewire is positioned within the desired location, a catheter or other tubular device may be positioned over the guidewire and used to convey other medical instruments into the desired blood vessel or duct.

Alternatively, a guiding catheter is used to negotiate the vasculature of a patient. One example of a guiding catheter is described in U.S. Pat. No. 4,898,577 to Badger et al. The Badger guiding catheter includes a single elongate shaft that has a deflectable distal portion controllable by a pull wire. Once the distal portion is at the required deflection or location within the patient, the guidewire or medical instrument is fed through the catheter.

The deflectable catheter is controlled at a proximal end of the catheter by a control handle that operates the pull wire to deflect the catheter, for example, as shown in U.S. Pat. No. 6,171,277. However, with conventional catheter steering mechanisms, it is sometimes difficult to accurately position the catheters in certain body vessels, such as branch veins. For instance, the mechanisms are awkward or require the use of two hands. Other steering mechanisms require pull wires to be wound and unwound around a rotatable cam wheel, causing increased fatigue on the pull wires, and potentially shortening the life of the device.

What is needed is a deflectable catheter that overcomes the shortcomings of previous deflectable catheters. What is further needed is a deflectable catheter that allows for more accurate positioning of the distal end of the deflectable catheter, and that is usable with a single hand.

### SUMMARY

A catheter assembly includes a handle assembly, and a catheter body coupled with the handle assembly, where the catheter body extends to a deflectable distal end, and the deflectable distal end is controllable by a flexible element. An actuator member is coupled with the flexible element, and movement of the actuator member provides for movement of the flexible element. The actuator has a locked mode and an operational mode, where the actuator and the flexible element are not movable relative to the handle assembly when the actuator is in the locked mode.

Several options for the catheter assembly are as follows. For instance, in one option, the actuator is in a first position in the locked mode and a second position in the operational mode, where the first position is different than the second position. In another option, the flexible element is longitudinally movable by a gearing mechanism coupled with the actuator. In yet another option, the catheter body includes a catheter body lumen, the handle assembly has a handle lumen, and the catheter body lumen is communicatively coupled with the handle lumen. The catheter assembly further includes, in another option, a spring, for instance, a living hinge, is coupled with the actuator, where the spring facilitates movement of the actuator between the locked mode and the operational mode. In yet another option, in the operational mode, the actuator is depressed toward a position within the handle assembly. In yet another option, a trigger is included with the catheter assembly, where movement of the trigger places the actuator member assembly in an operational mode.

In another embodiment, a catheter assembly includes a handle assembly, a catheter body coupled with the handle assembly, and a steering mechanism. The catheter body has a deflectable distal end controllable by a flexible element, where the steering mechanism controls movement of the deflectable distal end. The catheter assembly further includes an indicator associated with the steering mechanism, where the indicator provides affirmative feedback while the steering mechanism is in an operational mode.

Several options are as follows. For instance, in one option, the indicator provides an audible click when the steering mechanism is placed in the operational mode and/or the locked mode. In yet another option, the indicator provides tactile feedback when the steering mechanism is placed in the operational mode and/or the locked mode.

In another embodiment, a method comprises manipulating a catheter assembly, the catheter assembly including a handle assembly, a catheter body controllable by a flexible element coupled with the handle assembly. An actuator member is coupled with the flexible element, where movement of the actuator member provides for movement of the flexible element, and the actuator has a locked mode and an operational mode. The actuator and the flexible element are not movable relative to the handle assembly when the actuator is in the locked mode. The method further includes moving the actuator and unlocking the actuator, and placing the actuator in an operational mode. In addition, the method includes steering the catheter assembly including moving the actuator and deflecting the distal end.

Several options for the method are as follows. For instance, in one option, the method further includes releasing the actuator and locking movement of the flexible element relative to the handle assembly. In another option, the method further includes providing feedback to the user when the actuator is placed in the operational mode and/or the locked mode. In yet another option, placing the actuator in the locked mode includes enmeshing a gear coupled with the actuator with a static component, for example a paw as discussed above. Optionally, moving the actuator includes depressing the actuator within the handle assembly.

The deflectable catheter allows for single handed precise movement of the distal tip, and allows for locking a position in place easily. The lock further assists in preventing inadvertent movement of the distal tip, for example, during an ablation procedure. Furthermore, with the above-described design, the distal end can be easily configured to have different radius of curvature by varying the stroke length. Another option is to vary the input/output of the actuator by modifying the gear ratio. A further benefit of the device is the feedback provided when the lock is released, for example, the audible click. This affirmatively informs the physician when the steering mechanism is placed in the operational mode and/or the locked mode.

These and other embodiments, aspects, advantages, and features of the present invention will be set forth in part in the description which follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in the appended claims and their equivalents.

### BRIEF DESCRIPTION OF THE DRAWINGS

FIG. 1A is a perspective view illustrating a deflectable catheter assembly constructed in accordance with one embodiment.

FIG. 1B is a perspective view illustrating a deflectable catheter assembly constructed in accordance with one embodiment.

FIG. 2 is a perspective view illustrating a deflectable catheter body constructed in accordance with one embodiment.

FIG. 3 is a perspective view illustrating a distal portion of the deflectable catheter body constructed in accordance with one embodiment.

FIG. 4 is a perspective view illustrating a handle assembly constructed in accordance with one embodiment.

FIG. 5A is an exploded view illustrating a portion of a catheter assembly constructed in accordance with one embodiment.

FIG. 5B is an exploded view illustrating a portion of a catheter assembly constructed in accordance with another embodiment.

FIG. 6 is a side view illustrating a portion of the catheter assembly in a locked mode constructed in accordance with one embodiment.

FIG. 7 is a side view illustrating a portion of the catheter assembly in an operational mode constructed in accordance with one embodiment.

### DESCRIPTION OF THE EMBODIMENTS

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

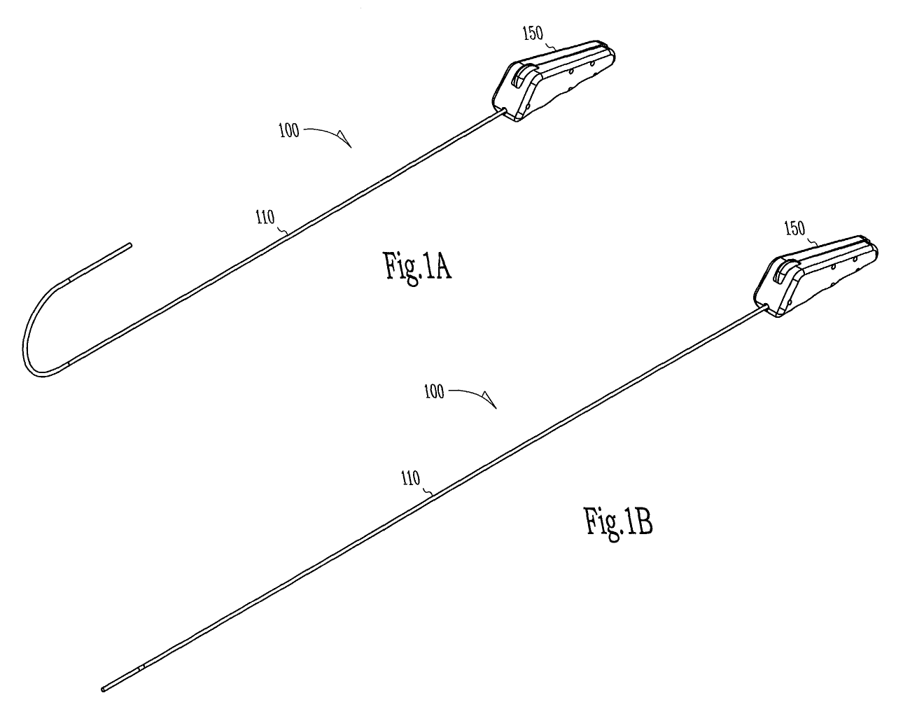

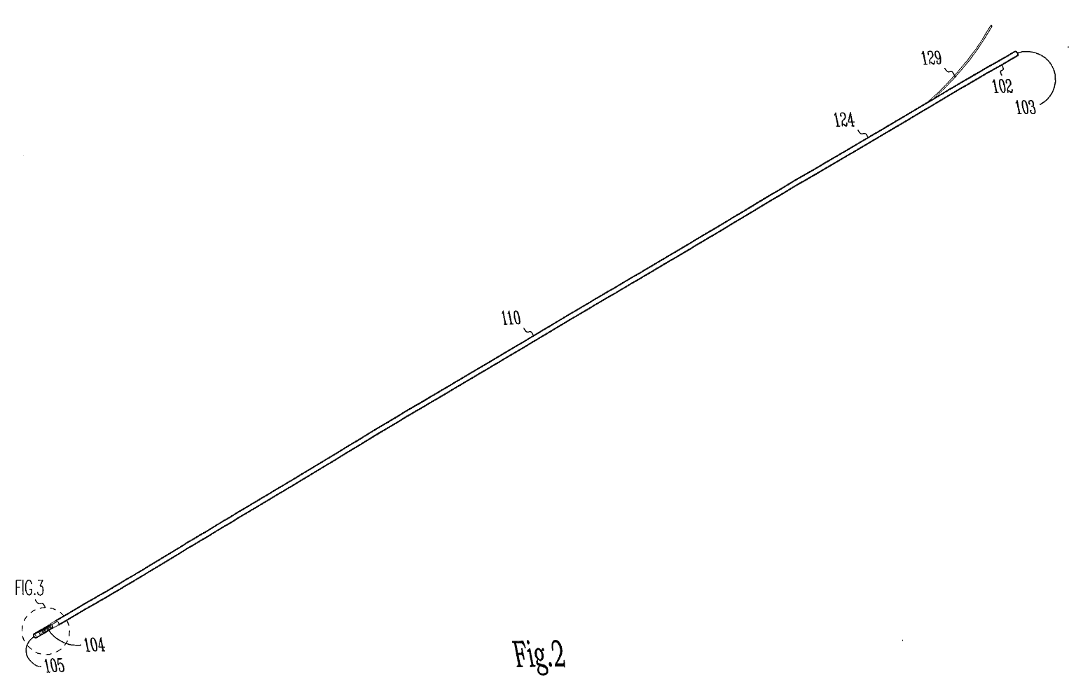

FIGS. 1A and 1B illustrate a deflectable catheter assembly 100, where FIG. 1A illustrates the deflectable catheter assembly 100 in an articulated position, and FIG. 1B illustrates the deflectable catheter assembly 100 in an unarticulated position. The deflectable catheter assembly 100 includes a deflectable catheter body 110 and a handle assembly 150 that houses steering mechanisms for deflection of the catheter body 110. The handle assembly 150, as described in greater detail below, allows for the deflection of a distal end of the catheter body 110. In one option, the pull wire 120 (FIG. 2) is connected to an actuator that is slid or rotated to apply tension to the pull wire 120 (FIG. 2). As tension is applied to the pull wire 120 (FIG. 2), the pull wire anchor at the distal end of the catheter body 110 is pulled which causes the distal portion of the catheter body 110 to curve in a predetermined direction or directions.

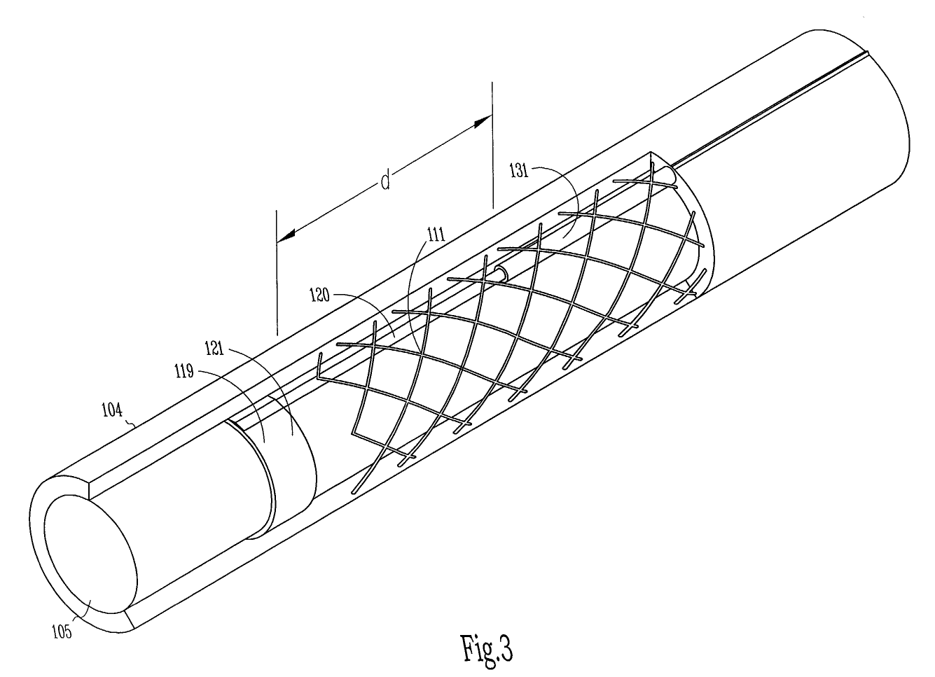

With reference to FIG. 2, the catheter body 110 comprises, in one option, an elongate tubular construction that is flexible yet substantially non-compressible along its length. The deflectable catheter body 110 extends from a proximal end 102 to a distal end 104, where the distal end 104 is disposed within a patient. At the proximal end 102 is a proximal tip 103, and at the distal end 104 is a distal tip 105. At the proximal end 102, the physician controls the deflection of the deflectable catheter body 110 with the handle assembly 150 (FIGS. 1A and 1B) and a pull wire 120 (FIG. 3), as further described below. The distal end 104 is deflected to traverse various branch vessels with the catheter assembly 100 (FIG. 1).

FIG. 3 illustrates a partial cut-away view of FIG. 2, including the distal end 104 of the catheter body 110. The catheter body 110 includes a pull wire anchor 121 that is secured to the catheter body 110. The pull wire 120 is mechanically secured to the pull wire anchor 121, for example, by welding the pull wire 120 to the pull wire anchor 121. It should be noted that the pull wire can be secured to the distal end 104 of the catheter body 110 in other manners. The pull wire anchor 121, in one option, comprises a marker band 119 that is viewable, for example, under fluoroscopy. In one option, the catheter body 110 includes a stiffening member embedded therein, such as a braided stainless steel member 111. The stiffening member facilitates rotation of the distal end 104 from the proximal end 102, and also assists in preventing the catheter body 110 from collapsing.

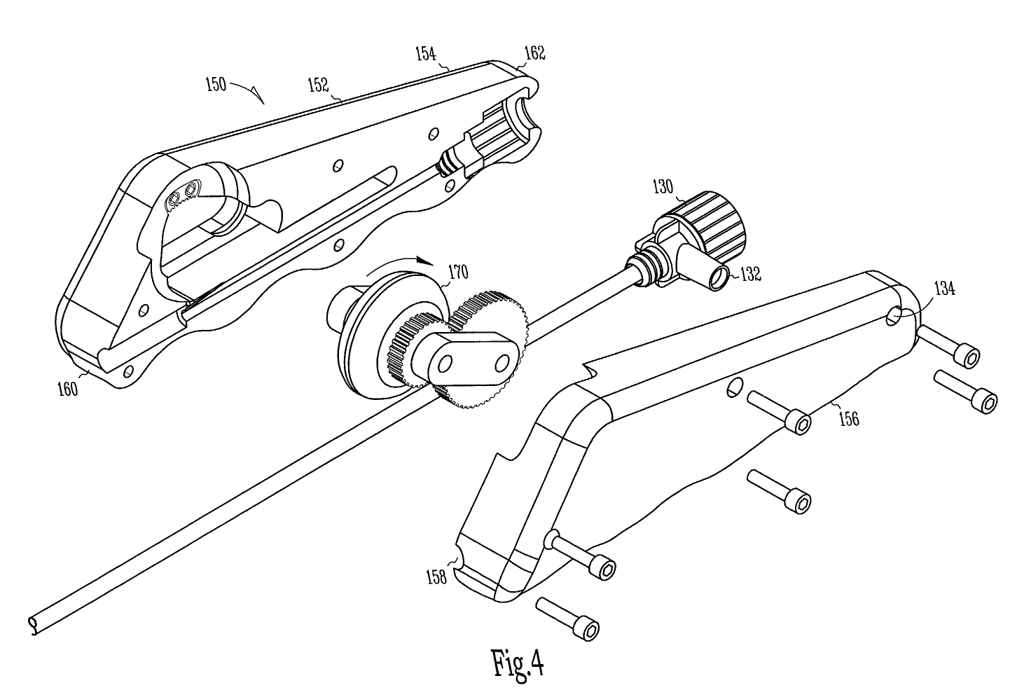

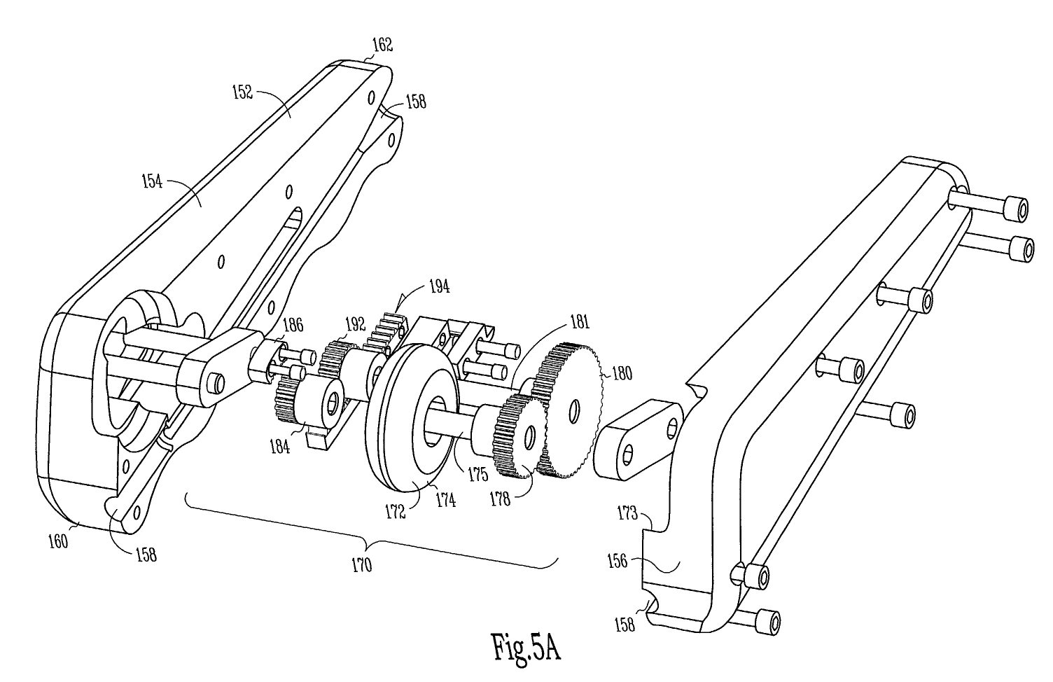

The handle assembly 150 is shown in greater detail in FIGS. 4, 5A, and 5B. The handle assembly 150 includes a handle housing 152 is designed to easily and comfortably fit into a practitioners hand, and to be easily manipulated with single hand use. In one option, the handle housing 152 are formed of a first portion 154 and a second portion 156 that are coupled together, for example, by one or more threaded fasteners. Other devices and/or methods for coupling the first and second portions 154, 156 of the handle housing 152 are suitable as well, such as, but not limited to, adhesive, welding, snap-fit, etc.

The handle housing 152 includes a handle lumen 158 therein. The handle lumen 158 is aligned with the delivery lumen of the catheter body 110 (FIG. 2), and the handle lumen 158 extends from a first end 160 to a second end 162 of the housing. A medical instrument, such as, but not limited to, a guidewire, a lead, an ablating device, etc., is disposed through the second end 162 of the housing 152 and through the delivery lumen of the catheter body 110 (FIG. 2). In one option, a valve 130 is coupled with the delivery lumen of the catheter body 110 (FIG. 2). The valve 130 provides further prevention of inadvertent fluid leakage from the delivery lumen. In another option, a side port 132 is coupled with the valve 130, which allows for the valve 130 to be flushed with fluids. The side port 132 is disposed through a portion of the handle assembly 150, for example, through an opening 134, allowing access to the side port 132 by a physician or medical technician. It should be noted that the valve 130 and/or the side port 132 can be combined with any of the above or below discussed embodiments.

The handle assembly 150 includes therein the actuator assembly 170 that moves the pull wire 120 (FIG. 2), and deflects the distal end 104 (FIG. 2) of the catheter body 110 (FIG. 2). Referring to FIG. 5A, the actuator assembly 170 includes an actuator 172 disposed through a slot 173 of the handle housing 152. The actuator 172 is manipulatable by an operator to deflect the distal end 104. In one option, the actuator 172 includes a wheel member 174 that is easily manipulated by the thumb in a rolling motion. The rotational input from the thumb is transferred into linear movement to provide the linear stroke for the pullwire on the proximal end, as further described below.

The following is one example of how to construct the steering assembly. It should be noted that several variations exist, including more simplified gearing configurations. In one option, the actuator 172 is coupled with a first axle 175 that rotates about a first actuator axis 176. Also coupled with the first axle 175 is a first gear 178 that meshes with a second gear 180. The second gear 180 is coupled with a second axle 181 that rotates about a second axis 182.

A third gear 184, in one option, is disposed on the opposite side of the actuator 172 as the first gear 178. The third gear 184 is fixed with the first axle 175 and is adapted to mesh with a static component, such as locking paw 186, when the actuator assembly 170 has been placed in a locked mode. The locking paw 186 is affixed to the handle portion, for example, with threaded fasteners. In one option, the locking paw 186 includes, for example, teeth integrally formed within the handle housing. A biasing member 202, such as, but not limited to, a spring, a living hinge, a spring steel member, biases the third gear 184 into meshing with the locking paw 186 when no force is placed on the actuator 172. It should be noted that one or more biasing members 202 can be used to bias the actuator 172 into a locked position.

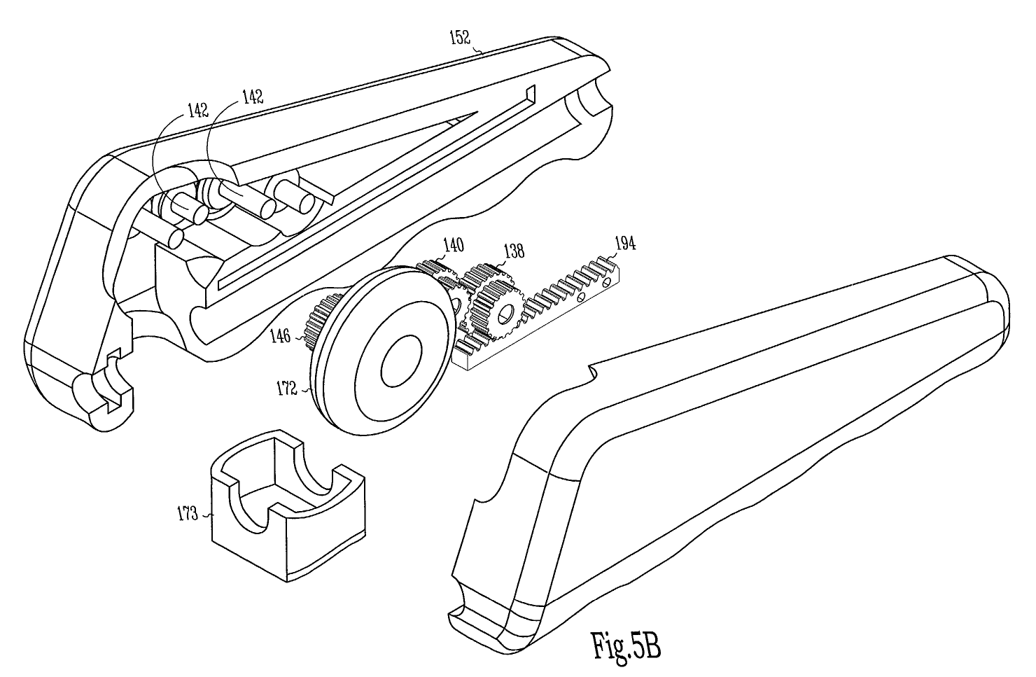

The locking paw 186 mechanically prevents the actuator 172 from moving until it is moved out of the locked mode. When the actuator assembly 170 is placed in the locked mode, as shown in FIG. 6, the actuator 172 is mechanically locked from moving, which also locks the pull wire from moving. This is particularly advantageous over conventional designs, or designs that prevent movement by friction, since the physician can be confident that the deflection of the distal end will not be inadvertently modified. Furthermore, when the distal end is in a highly articulated position, the distal end will not succumb to change when the physician releases the steering mechanism, for example, to introduce other instruments through the catheter assembly.

FIG. 5B illustrates another example of the gearing mechanism for the catheter assembly. The actuator 172 is coupled with a series of gears 138 that, in one option, are assembled in a linear fashion. The series of gears 138 are each coupled with an axle 142, about which each gear 140 rotates. In one option, the axle 142 includes a projection 144, such as a post, that extends from an inner surface 146 of the handle housing 152. Alternatively, or in combination, the axle 142 is disposed through or made integral with the gear 140, and the axle 142 is disposed within a recess formed within the handle housing 152. The axle 142 can be coupled directly or indirectly with the handle housing 152. The series of gears 138 cooperatively operate as is shown in FIG. 5B, where a first gear 146 is coupled with the actuator 172. Rotation of the actuator 172 causes rotation of the series of gears 138, and linear movement of the rack 194, as further discussed below. Locking and unlocking of the series of gears 138 can be achieved using the various embodiments discussed above and below, and shown in the Figures.

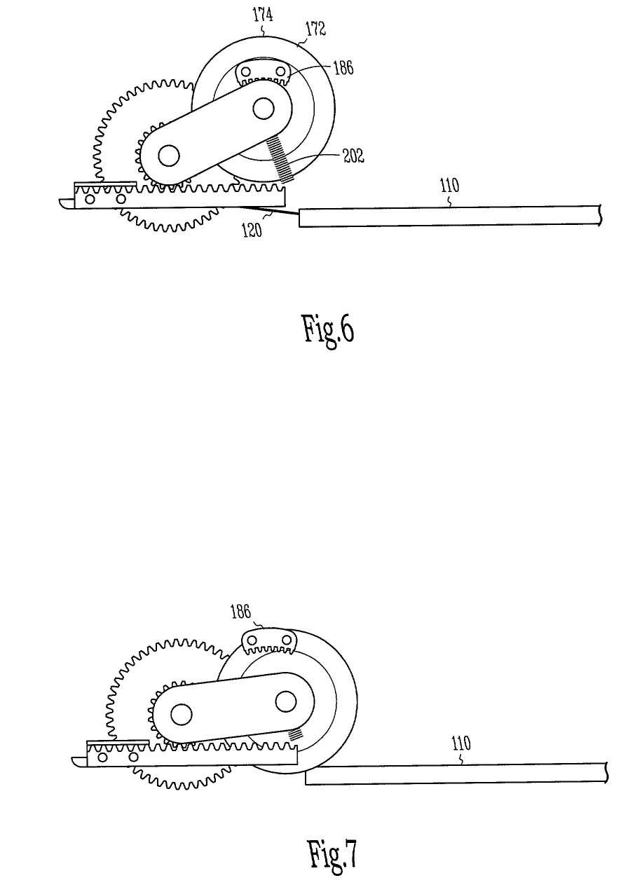

When the actuator assembly 170 is affirmatively placed in an operational mode, as shown in FIG. 7, the third gear 184, or the series of gears 138, is free to rotate, allowing the actuator 172 to freely rotate. In one option, to place the actuator assembly 170 in an operational mode, the actuator 172 is depressed to a position at least partially within the handle housing 152. As the actuator 172 is depressed, the user overcomes the bias from the biasing member, and the third gear 184 disengages from the locking paw 186. In another option, a trigger 173 (FIG. 5B) that is remote from the actuator 172 is used to place the actuator assembly 170 in operational mode. In one option, as the actuator assembly 170 is placed in operational mode, an audible click can be heard, thereby notifying the physician that the assembly is affirmatively in the operational mode. Other options for indicating to the physician that the status of operation has changed are possible as well. For example, a tactile click can be felt on the actuator 172, or a visual indicator can be provided, as further discussed below.

During operation, when the actuator assembly 170 is in the operation mode, the actuator 172 drives one or more gears 190, including a driving gear 192. The driving gear 192 drives a rack 194 that is coupled with the pull wire 120 (FIGS. 2 and 6). As the actuator 172 is moved, for example, rotated, the rack 194 is moved linearly and thereby pulls the pull wire 120. The linear movement of the rack 194 in combination with the pull wire 120 assists in preventing unnecessary fatigue being placed on the pull wire 120, for example by wrapping and unwrapping the pull wire 120 around a rotating member.

As the pull wire 120 is moved, this pulls on the pull wire anchor, and the distal end of the catheter body is deflected into position as desired by the physician. In one option, an indicator is associated with the movement or deflection of the catheter body, such that feedback is provided while the body is being moved. When in place or in a proper position, the physician releases the actuator 172, allowing the actuator assembly 170 to be locked in place, and further movement of the distal end is affirmatively prevented. In one option, as the physician releases the actuator 172, an audible click can be heard. For example, the meshing of the gears can be configured to cause an audible click. Other options for indicating to the physician that the status of operation has changed are possible as well. For example, a tactile click can be felt on the actuator 172, or a visual indicator can be provided when the actuator 172 is placed in the locked mode. In another option, the tactile click is caused by friction or rubbing of two or more components. The notice to the physician, in another option, involves an audible sound, or a tactile or visual indicator while the actuator 172 is being moved to manipulate the flexible element. For instance, in one option, while the actuator 172 is moved by the physician, a projection or finger would mesh with indentations or projections on the actuator 172, allowing the physician to feel or hear a clicking sound as the actuator 172 is rotated or moved. This can be done exclusive to the audible click or tactile click or visual indicator, or it can enhance these features. It should be noted that other indicators can be incorporated herein, and/or incorporated with the various embodiments discussed/shown above and below.

In another embodiment, a method comprises manipulating a catheter assembly, the catheter assembly including a handle assembly, a catheter body controllable by a flexible element coupled with the handle assembly. An actuator member is coupled with the flexible element, where movement of the actuator member provides for movement of the flexible element, and the actuator has a locked mode and an operational mode. The actuator and the flexible element are not movable relative to the handle assembly when the actuator is in the locked mode. The method further includes moving the actuator and unlocking the actuator, and placing the actuator in an operational mode. In addition, the method includes steering the catheter assembly including moving the actuator and deflecting the distal end.

Several options for the method are as follows. For instance, in one option, the method further includes releasing the actuator and locking movement of the flexible element relative to the handle assembly. In another option, the method further includes providing feedback to the user when the actuator is placed in the operational mode and/or the locked mode. In yet another option, placing the actuator in the locked mode includes enmeshing a gear coupled with the actuator with a static component. Optionally, moving the actuator includes depressing the actuator within the handle assembly.

Advantageously, the above-described deflectable catheter allows for increased control of the distal deflectable catheter end. The locking mechanism provides for accurate locking of the deflectable end in a certain position, allowing the physician increased control during the placement of the catheter within a patient. Furthermore, the indicator informs the physician when the deflectable catheter assembly has been placed in a locked and/or operational mode.

The deflectable catheter allows for single handed precise movement of the distal tip, and allows for locking a position in place easily. The lock further assists in preventing inadvertent movement of the distal tip, for example, during an ablation procedure. Furthermore, with the above-described design, the distal end can be easily configured to have different radius of curvature by varying the stroke length. Another option is to vary the input/output of the actuator by modifying the gear ratio. A further benefit of the device is the feedback provided when the lock is released, for example, the audible click. This affirmatively informs the physician when the steering mechanism is placed in the operational mode and/or the locked mode.

It is to be understood that the above description is intended to be illustrative, and not restrictive. Many other embodiments will be apparent to those of skill in the art upon reading and understanding the above description. It should be noted that embodiments discussed in different portions of the description or referred to in different drawings can be combined to form additional embodiments of the present invention. The scope of the invention should, therefore, be determined with reference to the appended claims, along with the full scope of equivalents to which such claims are entitled.

What is claimed is:

1. A catheter assembly comprising:
a handle assembly;
a catheter body coupled with the handle assembly, the catheter body extending to a deflectable distal end, the deflectable distal end controllable by a flexible element;
an actuator member coupled with the flexible element, and movement of the actuator member provides for movement of the flexible element; and
the actuator having a locked mode and an operational mode, where the actuator and the flexible element are not movable when the actuator is in the locked mode.

2. The catheter assembly as recited in claim 1, wherein the actuator is in a first position in the locked mode and a second position in the operational mode, where the first position is different than the second position.

3. The catheter assembly as recited in claim 1, wherein the flexible element is longitudinally movable by a gearing mechanism coupled with the actuator.

4. The catheter assembly as recited in claim 1, wherein the catheter body includes a catheter body lumen therein, the handle assembly having a handle lumen therein, the catheter body lumen communicatively coupled with the handle lumen.

5. The catheter assembly as recited in claim 1, further comprising a spring coupled with the actuator, where the spring facilitates movement of the actuator between the locked mode and the operational mode.

6. The catheter assembly as recited in claim 5, wherein the spring includes a living hinge.

7. The catheter assembly as recited in claim 1, wherein the actuator is depressed toward a position within the handle assembly when the actuator is in the operational mode.

8. The catheter assembly as recited in claim 1, further comprising at least one gear coupled with the flexible element, and the gear is enmeshed with a locking member when the actuator is in the locked mode.

9. The catheter assembly as recited in claim 1, further comprising a trigger associated with the handle assembly, where the trigger allows the actuator to transition between the locked mode and the operational mode.

10. The catheter assembly as recited in claim 1, where in the catheter body includes a delivery lumen, and the catheter assembly further includes a valve coupled with the delivery lumen.

11. A catheter assembly comprising:
a handle assembly
a catheter body coupled with the handle assembly;
the catheter body extending to a deflectable distal end controllable by a flexible element; and
an actuator member assembly coupled with the flexible element, and movement of the actuator member provides for movement of the flexible element, the actuator member assembly including a trigger, where movement of the trigger places the actuator member assembly in an operational mode.

12. The catheter assembly as recited in claim 11, wherein movement of the trigger places the actuator member assembly in a locked mode, and the flexible element is prevented from moving relative to the handle assembly.

13. The catheter assembly as recited in claim 11, wherein the handle assembly includes at least one gear coupled with the actuator member assembly, a rack is meshed with the gear, the rack is coupled with the flexible element, and where linear movement of the rack and the flexible element is converted to rotatable movement of the gear.

14. The catheter assembly as recited in claim 13, further comprising a locking mechanism comprising a paw that meshes with the gear and prevents movement of the gear.

15. The catheter assembly as recited in claim 11, further comprising means for providing feedback when the actuator member assembly is placed in the operational mode.

16. The catheter assembly as recited in claim 15, wherein the means for providing feedback comprises an audible click upon movement of the trigger.

17. The catheter assembly as recited in claim 11, wherein the actuator member assembly includes an actuator, and the trigger is associated with the handle assembly in a location remote from the actuator.

18. A catheter assembly comprising:
a handle assembly;
a catheter body coupled with the handle assembly, the catheter body extending to a deflectable distal end, the deflectable distal end controllable by a flexible element;
a steering mechanism coupled with the flexible element, the steering mechanism controlling movement of the deflectable distal end; and
an indicator associated with the steering mechanism, where the indicator provides affirmative feedback while the steering mechanism is in an operational mode.

19. The catheter assembly as recited in claim 18, wherein the indicator provides an audible click when the steering mechanism is placed in the operational mode.

20. The catheter assembly as recited in claim 18, wherein the indicator provides an audible click when the steering mechanism is placed in the locked mode.

21. The catheter assembly as recited in claim 18, wherein the indicator provides tactile feedback when a mode of the steering mechanism is modified.

22. A method comprising:
manipulating a catheter assembly, the catheter assembly including a handle assembly, a catheter body coupled with the handle assembly, the catheter body extending to a deflectable distal end, the deflectable distal end controllable by a flexible element, an actuator member coupled with the flexible element, and movement of the actuator member provides for movement of the flexible element, and the actuator having a locked mode and an operational mode, where the actuator and the flexible element are not movable when the actuator is in the locked mode;
moving the actuator and unlocking the actuator, including placing the actuator in an operational mode; and
steering the catheter assembly including moving the actuator and deflecting the distal end.

23. The method as recited in claim 22, further comprising releasing the actuator and locking movement of the flexible element relative to the handle assembly.

24. The method as recited in claim 22, further comprising providing feedback to the user when the actuator is placed in the operational mode.

25. The method as recited in claim 22, further comprising providing feedback to the user when the actuator is placed in the locked mode.

26. The method as recited in claim 22, wherein placing the actuator in the locked mode includes enmeshing a gear coupled with the actuator with a static component.

27. The method as recited in claim 22, wherein moving the actuator includes depressing the actuator within the handle assembly.
